(19) United States
(12) Patent Application Publication
(10) Pub. No.: US 2006/0074105 A1
(43) Pub. Date: Apr. 6, 2006

(54) Substituted quinoline and quinazoline inhibitors of quinone reductase 2

(75) Inventors: Roy W. Ware JR., Raleigh, NC (US); Lindsay A. Hinkley, Cary, NC (US); Klaas P. Hardeman, Laarne (BE); Matthew G. Jenks, Durham, NC (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Serenex, Inc., Durham, NC (US)

(21) Appl. No.: 11/228,833

(22) Filed: Sep. 16, 2005

Related U.S. Application Data
(60) Provisional application No. 60/611,471, filed on Sep. 20, 2004.

(51) Int. Cl. A61K 31/4709; C07D 401/02; C07D 215/38

(52) U.S. Cl. 514/313; 546/159

(57) ABSTRACT

The present invention provides composition and methods of inhibiting quinone reductase 2 (QR2). The methods are useful in the treatment of malaria and autoimmune diseases. The compositions of the invention comprise quinoline and quinazoline derivatives. The invention also provides methods for inhibiting the activity of QR2 by contacting the enzyme with one or more compositions of the invention.

### FIELD OF THE INVENTION

The present invention encompasses methods and compositions for the treatment of malaria and autoimmune diseases. More particularly, the invention provides quinoline and quinazoline derivatives and methods for their use in inhibiting quinone reductase-2.

### BACKGROUND OF THE INVENTION

Malaria remains one of the major killers of humans worldwide, threatening the lives of more than one-third of the world's population (National Institutes of Health Publication No. 02-7139, September 2002). Quinoline-containing antimalarial drugs (CQs), such as chloroquine, quinine, primaquine, and mefloquine, have historically been the leading chemotherapeutic weapons in the fight against malaria. Sadly, many species of Plasmodium, the protozoan parasite that causes malaria, have become resistant to these drugs. However, close analogues of chloroquine and certain chloroquine derivatives maintain activity against chloroquine-resistant strains, which indicates that the resistance mechanism does not involve any change to the target of this class of drug, but rather involves a compound-specific resistance (Kaschula et al., J. Med. Chem., 45, 3531-3539 (2002)). Therefore, the development of CQ analogues and derivatives as antimalarials is necessary to circumvent the problem of resistance.

The Plasmodium parasites are transferred to a human host via an infected mosquito, whereby the parasites multiply and ultimately occupy the red blood cells as a safe haven from the host's immune system (See Kemp et al., Annu. Rev. Microbiol. 41, 181-208 (1987); Weatherall et al., The Anaemia of Plasmodium falciparum Malaria, London (1993); Miller, Science 257, 36-37 (1992)). While it is known that CQs kill the parasites as they reside within the red blood cells, the mechanisms of action are not fully elucidated. However, CQs selectively inhibit quinone reductase 2 (QR2) in the red blood cells, and it is postulated that this inhibition creates an environment that is toxic to the Plasmodium parasites (Graves et al., Mol. Pharmacol., 62, 1364-1372 (2002).

In addition to their antimalarial actions, CQs have therapeutic value in the treatment of lupus erythematosus and rheumatoid arthritis (Rynes, British J. Rheumatology, 36, 799-805 (1997); Colman, Annu. Rev. Biochem. 52, 67-91 (1983) and references cited therein). The efficacy of CQs in the treatment of these diseases was discovered serendipitously following the prophylactic treatment of some 3-4 million soldiers for malaria in World War II (Beek et al., Dermatolo., 19, 1-11 (1971)). The CQs have become the parenteral drugs of choice for treating the cutaneous manifestations of lupus as well as a variety of other dermatoses. In arthritis, in responsive patients, long term treatment with CQs can bring about significant improvement of symptoms to complete remission. A major side effect and contraindication of CQs in the treatment of both conditions, however, is the development of retinopathy which can lead to blindness if unchecked (Beek et al., Dermatolo. 19, 1-11 (1971)), Rynes, British J. Rheumatology, 36, 799-805 (1997)). The cause of retinopathy is unknown as are the molecular mechanisms underlying the therapeutic actions of CQs in the treatment of lupus and arthritis. However, because efficacious CQs inhibit QR2, the development of other CQ derivatives that inhibit QR2 may provide for new therapies with reduced side effects.

### BRIEF SUMMARY OF THE INVENTION

Compositions and methods for inhibiting quinone reductase 2 (QR2) are provided. The compositions of the invention include the quinoline and quinazoline derivatives shown in Formulas 1-13. These compositions are useful for modulating the activity of QR2, and for treating diseases where the inhibition of QR2 is advantageous. Thus, the compositions and methods of the invention find use in the treatment of malaria and immune diseases. Accordingly, one or more compounds of the invention can be formulated into pharmaceutical compositions.

### DETAILED DESCRIPTION OF THE INVENTION

Compositions and methods for the inhibition of quinone reductase 2 (QR2) are provided. The compounds of the invention are quinoline and quinazoline derivatives as shown in Formulas 1-13. The compositions and methods are useful in the treatment of malaria and immune disorders.

QR2 and its homolog QR1 catalyze the metabolic detoxification of quinones, a large class of potentially toxic compounds found in all respiring plant and animal cells. If not reduced to the hydroquinone form by QR1 or QR2, quinones can participate in redox cycling and generate reactive oxygen species. The Plasmodium parasite creates further oxidative stress through its digestion of hemoglobin. QR1 is not present in red blood cells, suggesting that QR2 is responsible for the removal of reactive quinones in these cells. The malarial parasite P. falciparum is sensitive to oxidative stress (Green et al., Adaptation to Malaria, Gordon and Breach Publishers, Amsterdam (1997)) and inhibiting QR2 activity creates an oxidative environment can be lethal to the parasite.

The human amino acid sequence for QR2 (also known as NRH:quinone oxireductase 2) was described by Jaiswal (1994) J. Biol. Chem. 269:14502-08 and is given by Swiss-Prot accession number P16083, both of which are herein incorporated by reference. Quinone reductase 2 is a member of enzyme class 1-6.99. In some embodiments, the present invention provides methods of inhibiting QR2. The QR2 molecule inhibited in the method is preferably a mammalian QR2 enzyme such as, for example human QR2, although the QR2 may be from any source.

In some embodiments, the invention provides methods of inhibiting the activity of QR2, where the method involves contacting QR2 with a compound of Formulas 1-13. QR2 may be contacted with the compound in vitro. Alternatively, QR2 may be contacted with the compound in vivo, for example in a cell expressing QR2.

Compounds of the Formulas 1-13 are useful in the treatment of diseases where the inhibition of QR2 is therapeutically advantageous. Thus, the compositions and methods of the invention find use in methods of treating malaria and immune diseases. Accordingly, one or more compounds of the invention can be formulated into a pharmaceutical composition.

In some embodiments, the methods of the invention encompass the use of compounds of Formula (1) 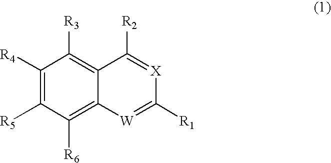
wherein W is N or N⁺O⁻
X is CR₁₄ or N
R₁ is H or trifluromethyl;
R₂ is NR₇R₈, OR₁₁, SR₁₂, or alkyl
R₃ is H or OR₁₃
R₄ is H or methoxy;
R₅ is H, Cl, or trifluoromethyl;
R₆ is H, NR₉R₁₀ or trifluoromethyl;
R₇ is H, C1-5 alkyl, cycloalkyl, heterocycloalkyl, aryl, heteroaryl, uredio, thioureido, alkenyl, alkynyl, amido, amino, alkoxy, alkylamino, alkylphosphonate, alkylnitrile, alkylhalo, or alkylhalo optionally substituted with C₁₋₅ alkyl, cycloalkyl, cycloalkenyl, heterocycloalkyl, C0-1 aryl, heteroaryl, alkenyl, alkynyl, amido, alkyoxy, alkylamino, alkylhydroxy, halo, hydroxyl, carboxyalte, allkylcarboxylate, acylazido, sulfonamide or alkyl halo;
R₈ is H, C1-5 alkyl, cycloalkyl, heterocycloalkyl, aryl, heteroaryl, uredio, thioureido, alkenyl, alkynyl, amido, amino, alkoxy, alkylamino, alkylphosphonate, alkylnitrile, alkylhalo, or alkylhalo optionally substituted with C₁₋₅ alkyl, cycloalkyl, cycloalkenyl, heterocycloalkyl, C0-1 aryl, heteroaryl, alkenyl, alkynyl, amido, alkyoxy, alkylamino, alkylhydroxy, halo, hydroxyl, carboxyalte, allkylcarboxylate, acylazido, sulfonamide or alkyl halo;
R₉ is H, C₁₋₅ alkyl, cycloalkyl, heterocycloalkyl, aryl, heteroaryl, alkylamino, alkylnitrile, or alkylphosphonate optionally substituted with C₁₋₅ alkyl, cycloalkyl, heterocycloalkyl, aryl, heteroaryl or alkylamino;
R₁₀ is H, C₁₋₅ alkyl, cycloalkyl, heterocycloalkyl, aryl, heteroaryl, alkylamino, alkylnitrile, or alkylphosphonate optionally substituted with C₁₋₅ alkyl, cycloalkyl, heterocycloalkyl, aryl, heteroaryl or alkylamino;
R₁₁ is alkyl, aryl or heteroaryl optionally substituted with alkyl, haloalkyl, aryl, or heteroaryl;
R₁₂ is alkyl, aryl or heteroaryl optionally substituted with alkyl, haloalkyl, aryl, or heteroaryl;
R₁₃ is alkyl or aryl optionally substituted with alkyl or haloalkyl; and
R₁₄is H or aryl.

In particular embodiments, the compounds of the invention include those encompassed by Formula (2): 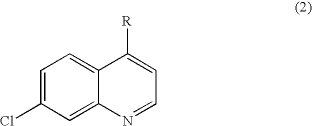
where R is selected from the group consisting of (3-methoxyphenyl)methylamino (2-1), (4,4-diethoxy)butylamino (2-2), isopentylamino (2-3), 2-(pyridin-2-yl)ethylamino (2-4), 2-(4-hydroxyphenyl)ethylamino (2-5), (2-chloro-4-fluoro)benzylamino (2-6), (pyridin-3-yl)methylamino (2-7), 3-(dibutylamino)propylamino (2-8), 2-(4-(ethoxycarbonyl)phenyl) ethylamino (2-9), 4-(hydrazinocarbonyl)phenylamino (2-10), naphthalen-1-ylmethylamino (2-11), 2,2-diphenylpropylamino (2-12), ((1r, 4r)-4-(ethoxycarbonyl)cyclohexyl)methylamino (2-13), 3-chloropropylamino (2-14), allylamino (2-15), prop-2-ynylamino (2-16), pyridin-2-ylmethylamino (2-17), (2,2-dimethyl-3-dimethylamino)propylamino (2-18), o-N-toluidinoamino (2-19), 4-(morpholino)phenylamino (2-20), benzylamino (2-21), 2-(acetamido)ethylamino (2-22), 1-(pyridin-2-yl)ethylamino (2-23), 2-(4-sulfamoylphenyl)ethylamino (2-24), (1-hydroxy-3-methyl)but-2-ylamino (2-25), (thiophen-2-ylmethyl)amino (2-26), morpholinoamino (2-27), 1,2,3,4-tetrahydronaphthalen-1-ylamino (2-28), 3a, 7a-dihydro-1H-benzo[d]imidazol-2-ylamino (2-29), (3-methoxyphenylethyl)amino (2-30), 2-(cyclohexenyl)ethylamino (2-31), (6-methylpyridin-2-yl)methoxy (2-32), (pyridin-2-ylmethyl)thio (2-33), pyridin-2-ylmethoxy (2-34), N-methyl-N-(pyridin-2-ylmethyl)amino (2-35), 1-(pyridin-2-yl)ethoxy (2-36), 3-(2-carboxy)naphthylamino (2-37), 4-(2,4-dichlorophenyl)thiosemicarbazido (2-38), and methylamino (2-39).

The structures of these compounds are shown below:

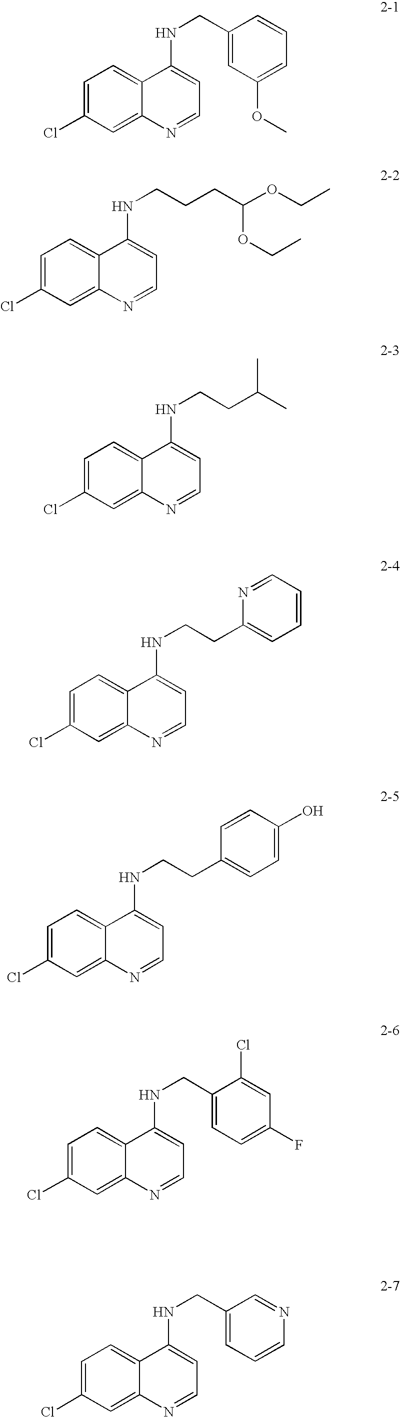

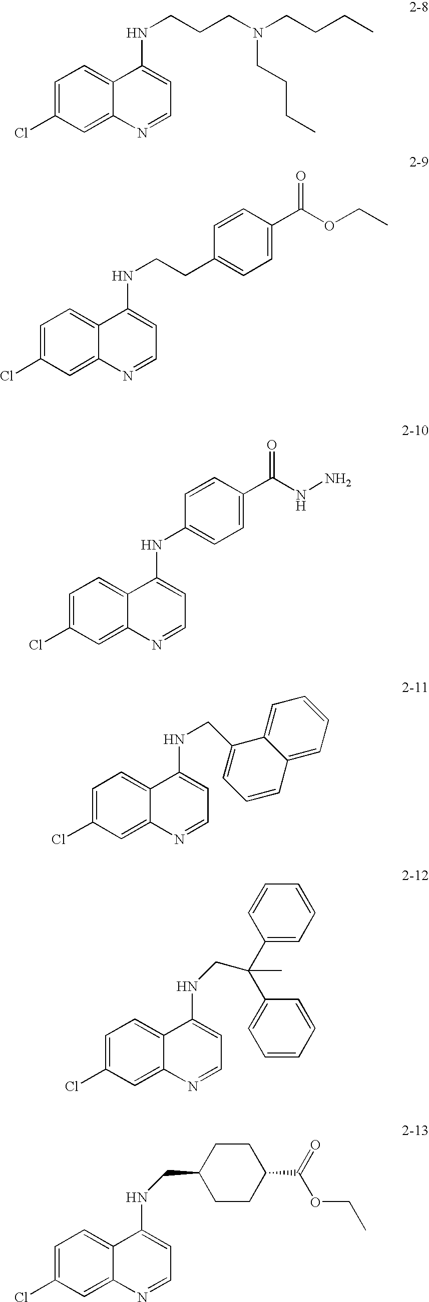

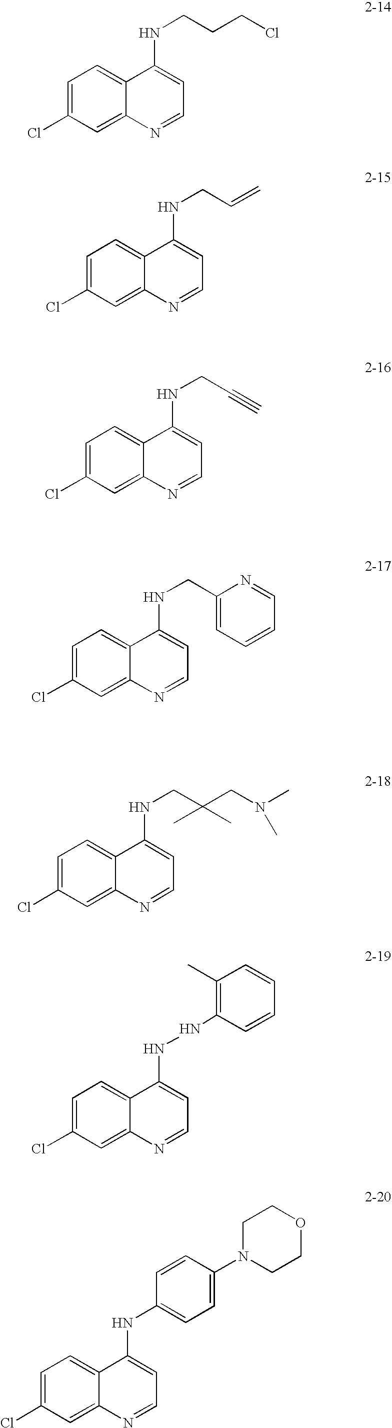

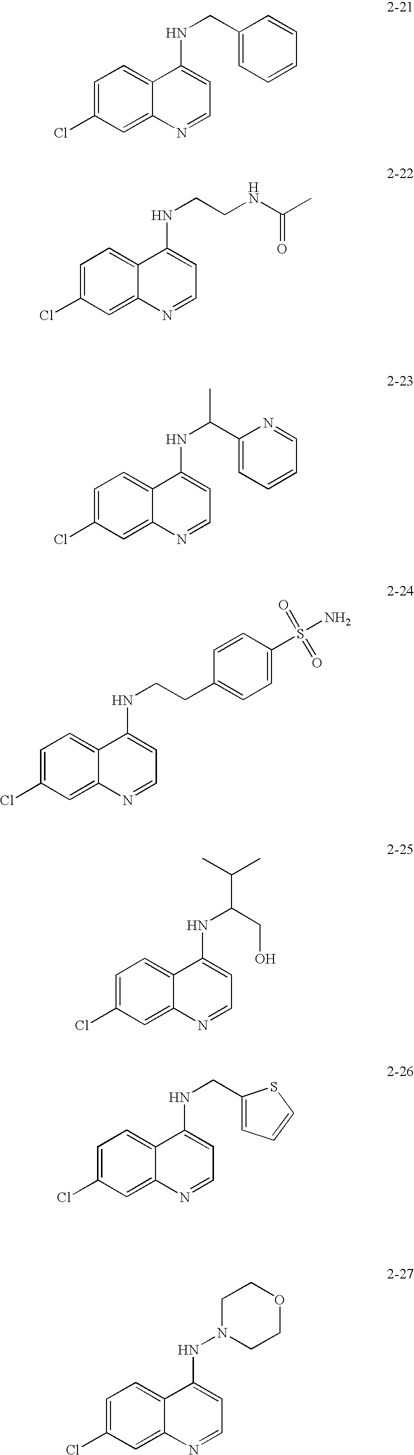

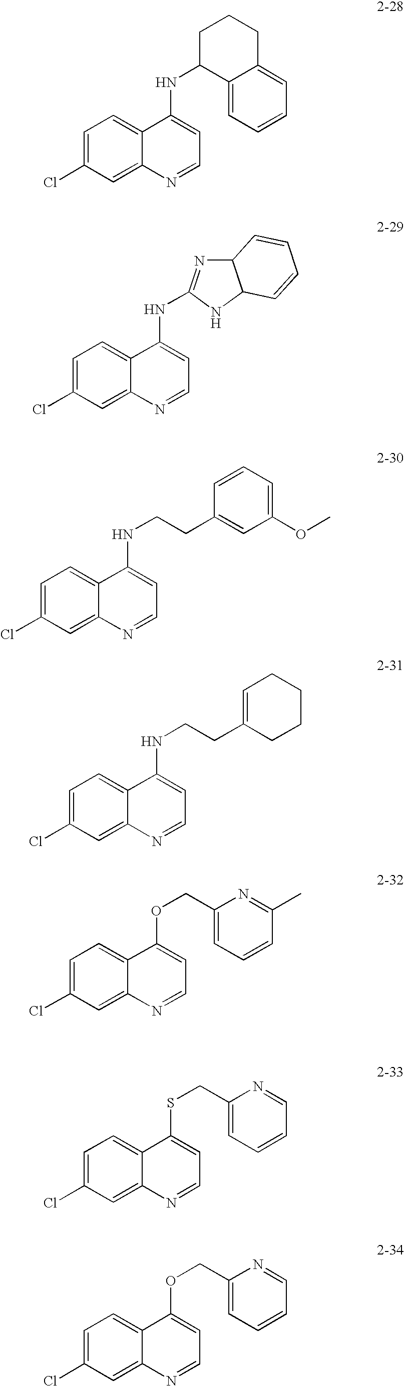

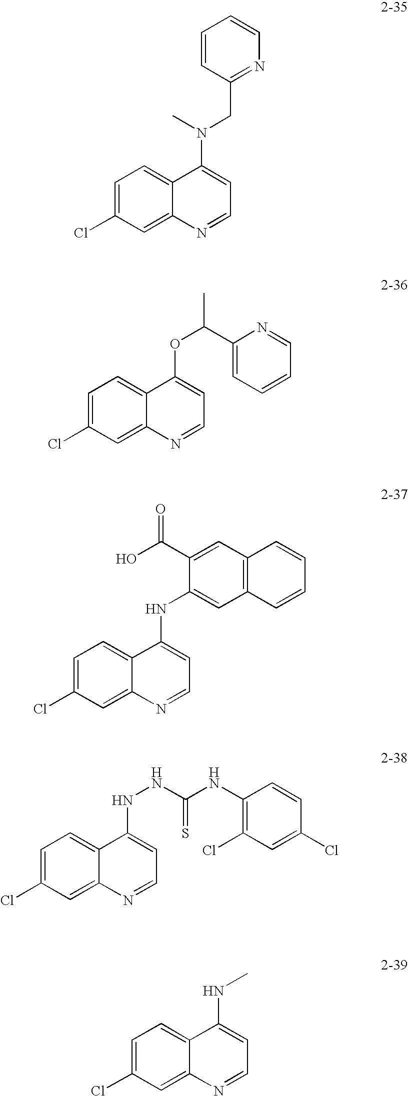

Other compounds that are useful in the methods of the invention include compounds of Formula (3): 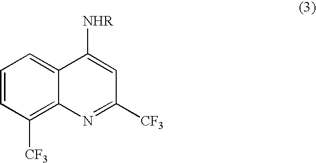
where R is selected from the group consisting of 2-(pyrrolidin-1-yl)ethyl (3-1), 2-(4-hydroxyphenyl)ethyl (3-2), 2-(2-hydroxypropylamino)ethyl (3-3), 3-(bis(2-hydroxyethyl)amino)propyl (3-4), 1-benzylpiperidin-4-yl (3-5), 2-(thiophen-2-yl)ethyl (3-6), 1-(4-fluorophenyl)ethyl (3-7) and 2-(pyridin-2-yl)ethyl (3-8).

The corresponding chemical structures are:

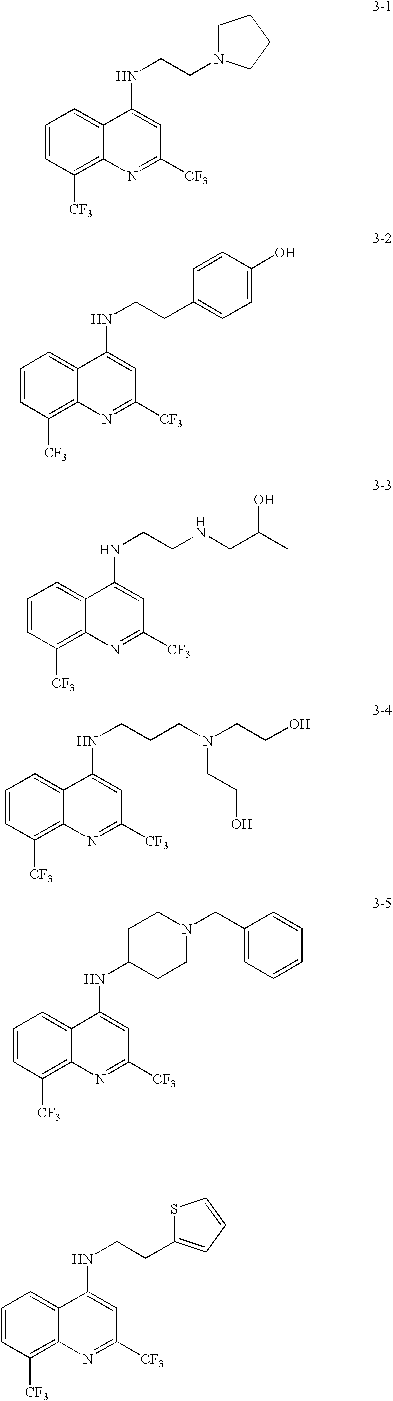

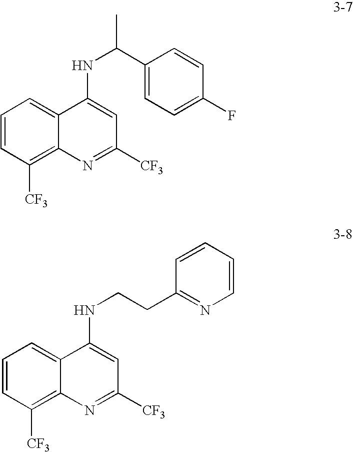

Other compounds that are useful in the methods of the invention include compounds of Formula (4): 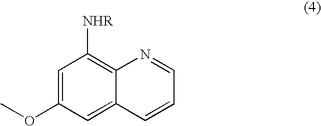
where R is selected from the group consisting of pyridin-2-ylmethyl (4-1), 1-benzylpiperidin-4-yl (4-2), 4-cyano-2,2-diethylbutyl (4-3), 2-chlorocyclopentyl (4-4), 4-(diethylamino)butan-2-yl (4-5), 2-(diethylphosphoryl)-1-methylethyl (4-6), 1-cyclopropylethyl (4-7), 1-ethylpiperidin-4-yl (4-8), 5-amino-2,2-diethylpentyl (4-9), 1-(furan-2-yl)ethyl (4-10).

The corresponding chemical structures are:

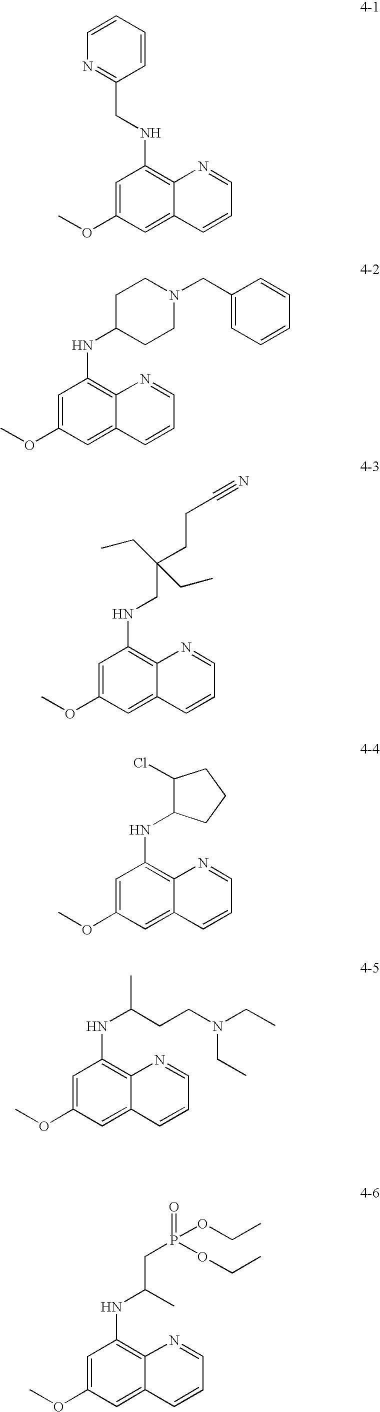

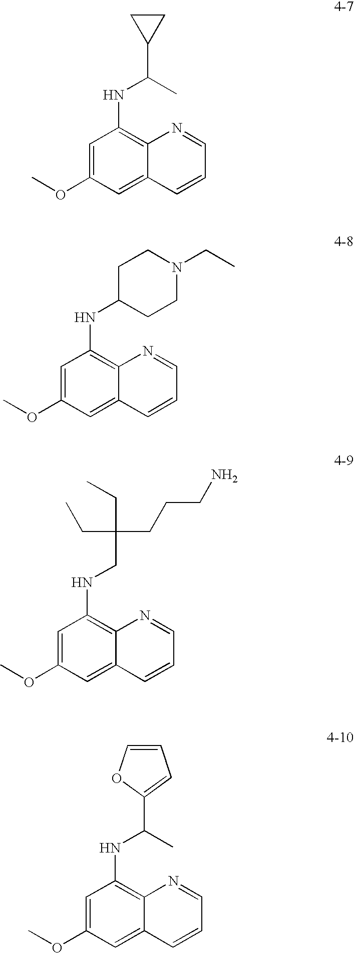

Another compound that is useful in the methods of the invention is the compound of Formula (5): N-(2-(5-nitropyridin-2-ylamino)ethyl)-2,7-bis(trifluoromethyl)quinolin-4-amine (5).

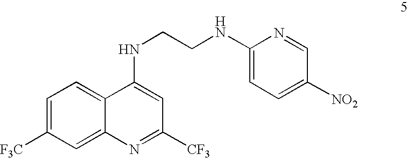

Other compounds useful in the methods of the invention include those of Formula (6) (7-chloro-N-(pyridin-2-ylmethyl)quinazolin-4-amine, Formula (7) (7-chloro-3-phenylquinoline), Formula (8) (N,N-dimethyl-7-(trifluoromethyl)quinolin-4-amine (8)), Formula (9) (6-methoxyquinoline N-oxide), and Formula (10) (4-amino-8-(trifluoromethyl)quinoline). The corresponding structures are shown below.

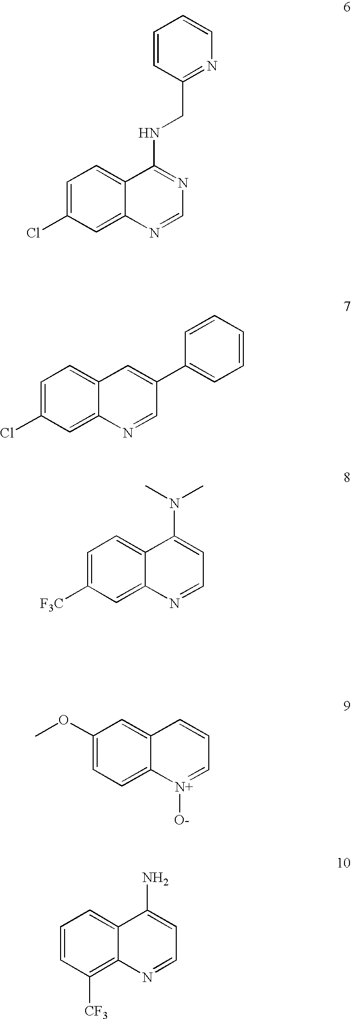

Other compounds that are useful in the methods of the invention include those encompassed by Formula (11): 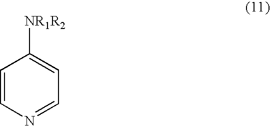
wherein R₁ is H, alkyl, aryl or heteroaryl optionally substituted with aryl or heteroaryl and R₂ is H, alkyl, aryl or heteroaryl optionally substituted with aryl or heteroaryl.

Other compounds that are useful in the methods of the invention include compounds of Formula (12): 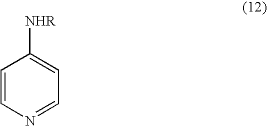
wherein R is a Cl alkyl optionally substituted with aryl or heteroaryl. In particular embodiments, R is selected from the group consisting of thiophen-2-ylmethyl (12-1), furan-2-ylmethyl (12-2), pyridin-3-ylmethyl (12-3) and pyridin-4-ylmethyl (12-4).

The corresponding chemical structures of this embodiment of the invention are:

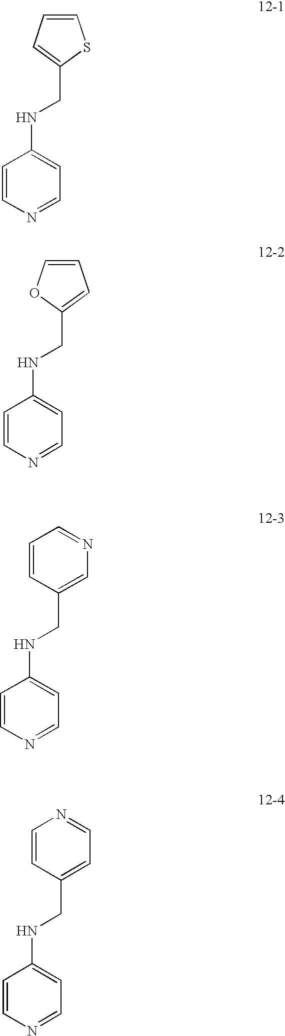

The invention also encompasses compositions comprising quinone and quinoline derivatives. In some embodiments, the compositions comprise compounds having Formula (13): 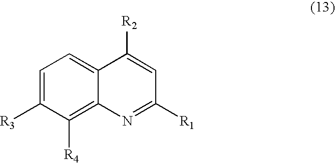
wherein R₁ is H or trifluromethyl;
R₂ is NHR₅, NR₅R₆, OR₅, SR₅
R₃ is H, Cl, or trifluromethyl
R₄ is H or trifluromethyl
R₅ is alkyl, allyl, propargyl, aryl, substituted aryl, heteroaryl, substituted heteroaryl, cycloalkyl optionally substituted with aryl, substituted or nonsubstituted heterocycloalkyl, C₁₋₂ alkyl optionally substituted with aryl, C₁₋₂ alkyl optionally substituted with mono- or di-substituted aryl, C₁₋₂ alkyl optionally substituted with substituted or nonsubstituted heteroaryl, C₁₋₂ alkyl optionally substituted with substituted or nonsubstituted cycloalkyl, C₁₋₂ alkyl optionally substituted with substituted or nonsubstituted heterocycloalkyl, C₁₋₃ alkyl optionally substituted with amionoalkyl, amido, aminoalkoxy, aminoheteroaryl, hydroxy, alkoxy, C₁₋₃ alkyamino optionally substituted with C₁₋₃ hydroxy.
R₆ is methyl.

In particular embodiments, the compositions of the invention comprise one or more of the compounds shown in formulas 2-1, 2-2, 2-3, 2-4, 2-5, 2-6, 2-7, 2-8, 2-9, 2-10, 2-11, 2-13, 2-15, 2-16, 2-17, 2-19, 2-20, 2-21, 2-22, 2-23, 2-24, 2-25, 2-26, 2-28, 2-29, 2-30, 2-32, 2-33, 2-34, 2-35, 2-36, 3-1, 3-2, 3-3, 3-4, 3-5, 3-6, 3-7, and 3-8.

In other embodiments, the compositions of the invention comprise compounds having Formula (4), (5), or (12). In particular embodiments, the compositions comprise the compounds shown in formulas 4-1, 4-2, 4-3, 4-4, 4-5, 4-6, 4-7, 4-8, 4-9, 4-10, 12-2, 12-3, and 12-4.

The compositions of the invention may be synthesized according to the methods described in Egan et al. (2000) J. Med. Chem. 43:283-291 and Stocks et al. (2002) J. Med. Chem. 45:4975-83.

The present invention includes all enantiomeric and diastereomeric forms of the compounds of Formulas 1-13 either individually or admixed in any proportions. The present invention further includes the use of prodrugs and active metabolites of the compounds of Formulas 1-13. A prodrug includes any compound which, when administered to a mammal, is converted in whole or in part to a compound of Formulas 1-13. An active metabolite is a physiologically active compound which results from the metabolism of a compound of Formulas 1-13, or a prodrug thereof, when such compound or prodrug is administered to a mammal.

The compounds of Formulas 1-13 above and their pharmaceutically acceptable esters, amides, salts, or solvates are sometimes hereinafter referred to as “the compounds according to the invention”.

The term “alkenyl” as used herein is intended to mean straight or branched chain unsaturated aliphatic hydrocarbons having one or more double bonds.

The term “alkyl” as used herein is intended to mean straight or branched chain alkyl. The term “C₁₋₅ alkyl” as used herein is intended to mean straight or branched chain alkyl of 1-5 carbon atoms.

The term “alkynyl” as used herein is intended to mean straight or branched chain unsaturated aliphatic hydrocarbons having one or more triple bonds.

The term “aryl,” alone or in combination, is intended to mean a monocyclic or polycyclic aromatic group with between 5 and 14 atoms in the ring.

The term “cycloalkyl” as used herein is intended to include monocyclic or fused polycyclic C₃-C₁₀ aliphatic hydrocarbon groups.

The term “alkyl halo” as used herein is intended to mean an alkyl group substituted with one or more halo substituents, either F, Cl, Br, or I, or combinations thereof.

The term “halogen” or “halo” as used herein is intended to mean F, Cl, Br, or I.

The term “heteroaryl” as used herein is intended to mean a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, aryl, haloaryl, nitro, amino, alkylamino, acylamino, alkylthio, alkylsulfinyl, alkylsulfonyl, arylsulfonyl, and cyano.

The term “heterocycloalkyl” as used herein is intended to mean monocyclic or fused polycyclic C₃-C₁₀ aliphatic hydrocarbon groups containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, aryl, haloaryl, nitro, amino, alkylamino, acylamino, alkylthio, alkylsulfinyl, alkylsulfonyl, arylsulfonyl, and cyano.

Quinoline and quinazoline derivatives generally provided in Formulas 1-13 and their esters, amides, salts, and solvates may be prepared in any manner known in the art for the preparation of compounds of analogous structure. In particular, the compounds can be prepared according to the methods described in Egan et al., J. Med. Chem., 43, 283-291 (2000) and Stocks et al., J. Med. Chem., 45, 4975-4983 (2002). Esters, amides, salts, solvates, prodrugs, and other derivatives of the compounds of the present invention may be prepared according to methods generally known in the art, such as, for example, those methods described by J. March, Advanced Organic Chemistry: Reactions, Mechanisms and Structure, 4^{th} Ed. (New York: Wiley-Interscience, 1992).

The compounds of the invention find use in inhibiting QR2. The primary function of QR2 and its homolog QR1 is to catalyze the metabolic detoxification of quinones, a large class of potentially toxic compounds found in all respiring plant and animal cells. If not reduced to the hydroquinone form by QR1 or QR2, quinones can participate in redox cycling and generate oxygen species. The compounds of the invention are quinoline and quinazoline derivatives. Compounds of the Formulas 1-13 are useful in the treatment of diseases where the inhibition of QR2 is advantageous. Thus, the compositions and methods of the invention find use in the treatment of malaria as well as autoimmune diseases. In this manner, at least one compound of the invention can be formulated into pharmaceutical compositions.

While the present invention is not limited to any particular mechanism of action, the inhibition QR2 allows for the build up of oxidative species, including quinones, in the red blood cells. It is these oxidative species which are believed to be toxic to the Plasmodium parasite. Thus, the compounds of the invention, as inhibitors of QR2, are useful for the treatment of malaria. The compounds of the invention also find use in the treatment of autoimmune diseases. Such autoimmune diseases include, but are not limited to, lupus (both systemic lupus erythematosus and lupus nephritis); psoriasis; scleroderma; CREST syndrome; inflammatory myositis; Sjogren's syndrome; mixed connective tissue disease; rheumatoid arthritis; psoriatic arthritis; palindromic rheumatism; eosinophilic fasciitis; dermatomyositis; juvenile chronic arthritis, erosive osteoarthritis; calcium pyrophosphate crystal deposition disease; multiple sclerosis; inflammatory bowel disease; colitis; Crohn's disease; acute respiratory distress syndrome; pulmonary inflammation; idiopathic pulmonary fibrosis; osteoporosis; delayed hypersensitivity; autoimmune thyroiditis; Hashimoto's disease; Grave's disease; asthma; primary biliary cirrhosis; idiopathic thrombocytopenic purpura; diabetes; leucopenia; opportunistic infections; thrombus formation; arteriosclerosis; therapy-induced diseases such as antibiotic allergy, gene vector hypersensitivity, and chemotherapy-induced human anti-mouse antibody induction; and neurological disease such as pathogenic neural cell apoptosis, Parkinson's disease, Alzheimer's, Huntington disease, and spinocerebellar ataxis/atrophies.

It is recognized that the compounds may be used alone or in combination for used in the methods of the invention. That is, one, two, three or any combination can be sued in the method. Likewise, they may be administered in one pharmaceutical composition, concomitant or sequentially in more than one pharmaceutical composition. In the same manner, they can be used with known compounds in treatments regimens.

Examples of pharmaceutically acceptable salts of the compounds according to the invention include acid addition salts. Salts of non-pharmaceutically acceptable acids, however, may be useful, for example, in the preparation and purification of the compounds. Suitable acid addition salts according to the present invention include organic and inorganic acids. Preferred salts include those formed from hydrochloric, hydrobromic, sulfuric, phosphoric, citric, tartaric, lactic, pyruvic, acetic, succinic, fumaric, maleic, oxaloacetic, methanesulfonic, ethanesulfonic, p-toluenesulfonic, benzesulfonic, and isethionic acids. Other useful acid addition salts include propionic acid, glycolic acid, oxalic acid, malic acid, malonic acid, benzoic acid, cinnamic acid, mandelic acid, salicylic acid, and the like.

An acid addition salt may be reconverted to the free base by treatment with a suitable base. Preparation of basic salts of acid moieties which may be present on a compound of the present invention may be prepared in a similar manner using a pharmaceutically acceptable base, such as sodium hydroxide, potassium hydroxide, ammonium hydroxide, calcium hydroxide, triethylamine, or the like. Esters of the compounds of the present invention may be prepared through functionalization of hydroxyl and/or carboxyl groups that may be present within the molecular structure of the compound. Amides and prodrugs may also be prepared using techniques known to those skilled in the art. For example, amides may be prepared from esters, using suitable amine reactants, or they may be prepared from anhydride or an acid chloride by reaction with ammonia or a lower alkyl amine. Prodrugs are typically prepared by covalent attachment of a moiety, which results in a compound that is therapeutically inactive until modified by an individual's metabolic system. Formulations While it is possible for the compounds of the present invention to be administered in the raw chemical form, it is preferred for the compounds to be delivered as a pharmaceutical formulation. Accordingly, there are provided by the present invention pharmaceutical compositions comprising at least quinoline or quinazoine derivative. As such, the formulations of the present invention comprise a compound of Formula 1-13, as described above, or a pharmaceutically acceptable ester, amide, salt, or solvate thereof, together with one or more pharmaceutically acceptable carriers therefore, and optionally, other therapeutic ingredients.

By “pharmaceutically acceptable carrier” is intended a carrier that is conventionally used in the art to facilitate the storage, administration, and/or the healing effect of the agent. Carriers should be acceptable in that they are compatible with any other ingredients of the formulation and not harmful to the recipient thereof. A carrier may also reduce any undesirable side effects of the agent. Such carriers are known in the art. See, Wang et al. (1980) J. Parent. Drug Assn. 34(6):452-462, herein incorporated by reference in its entirety.

Formulations of the present invention may include short-term, rapid-onset, rapid-offset, controlled release, sustained release, delayed release, and pulsatile release formulations, providing the formulations achieve administration of a compound as described herein. See Remington's Pharmaceutical Sciences (18^{th} ed.; Mack Publishing Company, Eaton, Pa., 1990), herein incorporated by reference in its entirety.

Pharmaceutical formulations according to the present invention are suitable for various modes of delivery, including oral, parenteral (including intravenous, intramuscular, subcutaneous, intradermal, and transdermal), topical (including dermal, buccal, and sublingual), and rectal administration. The most useful and/or beneficial mode of administration can vary, especially depending upon the condition of the recipient and the disorder being treated.

The pharmaceutical formulations may be conveniently made available in a unit dosage form, whereby such formulations may be prepared by any of the methods generally known in the pharmaceutical arts. Generally speaking, such methods of preparation comprise combining (by various methods) an active agent, such as the compounds of Formula 1-13 according to the present invention (or a pharmaceutically acceptable ester, amide, salt, or solvate thereof) with a suitable carrier or other adjuvant, which may consist of one or more ingredients. The combination of the active ingredient with the one or more adjuvants is then physically treated to present the formulation in a suitable form for delivery (e.g. shaping into a tablet or forming an aqueous suspension).

Pharmaceutical formulations according to the present invention suitable as oral dosage may take various forms, such as tablets, capsules, caplets, and wafers (including rapidly dissolving or effervescing), each containing a predetermined amount of the active agent. The formulations may also be in the form of a powder or granules, a solution or suspension in an aqueous or non-aqueous liquid, and as a liquid emulsion (oil-in-water and water-in-oil). The active agent may also be delivered as a bolus, electuary, or paste. It is generally understood that methods of preparations of the above dosage forms are generally known in the art, and any such method would be suitable for the preparation of the respective dosage forms for use in delivery of the compounds according to the present invention.

A tablet containing a compound according to the present invention may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agent.

Adjuvants or accessory ingredients for use in the formulations of the present invention can include any pharmaceutical ingredient commonly deemed acceptable in the art, such as binders, fillers, lubricants, disintegrants, diluents, surfactants, stabilizers, preservatives, flavoring and coloring agents, and the like. Binders are generally used to facilitate cohesiveness of the tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluoses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the formulation according to the present invention may be anionic, cationic, amphoteric, or nonionic surface active agents. Stabilizers may be included in the formulations to inhibit or lessen reactions leading to decomposition of the active agent, such as oxidative reactions.

Solid dosage forms may be formulated so as to provide a delayed release of the active agent, such as by application of a coating. Delayed release coatings are known in the art, and dosage forms containing such may be prepared by any known suitable method. Such methods generally include that, after preparation of the solid dosage form (e.g. a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the active agent over a prolonged period of time), and may also be delayed release. Sustained release formulations are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

Formulations for parenteral administration include aqueous and non-aqueous sterile injectable solutions, which may further contain additional agents, such as anti-oxidants, buffers, bacteriostats, and solutes, which render the formulations isotonic with the blood of the intended recipient. The formulations may include aqueous and non-aqueous sterile suspensions, which may contain suspending agents and thickening agents. Such formulations for parenteral administration may be presented in unit-dose or multi-dose containers, such as, for example, sealed ampoules and vials, and may be stored in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

The compounds according to the present invention may also be administered transdermally, wherein the active agent is incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agent is contained in a layer separate from the adhesive layer. Both types of patches also generally contain a backing layer and a liner that is removed prior to attachment to the skin of the recipient. Transdermal drug delivery patches may also be comprised of a reservoir underlying the backing layer that is separated from the skin of the recipient by a semi-permeable membrane and adhesive layer. Transdermal drug delivery may occur through passive diffusion or may be facilitated using electrotransport or iontophoresis.

Topical formulations may be in any form suitable and readily known in the art for delivery of an active agent to the body surface, including dermally, buccally, and sublingually. Typical examples of topical formulations include ointments, creams, gels, pastes, and solutions. Formulations for topical administration in the mouth also include lozenges.

Preferred unit dosage formulations are those containing a therapeutically effective amount, or an appropriate fraction thereof, of the active agent of the present invention. The term therapeutically effective amount, as used herein, is meant to refer to an amount effective to treat the disease of interest, such as cancer. Treatment can mean having a direct effect on an area in need of treatment, such as a tumor, or having a peripheral effect, such as through the activation or inhibition of a therapeutically associated enzyme.

In some embodiments, the pharmaceutical compositions of the present invention may comprise one or more compounds of the invention and an additional therapeutic agent useful for the treatment of malaria or arthritis. For example, in some embodiments the pharmaceutical compositions comprise a compound of Formulas 1-13 and an anti-malarial therapeutic agent such as, for example, chloroquine, quinine, quinidine, mefloquine, atovaquone, or artemisinin. In other embodiments, the pharmaceutical compositions comprise a compound of Formulas 1-13 and a second therapeutic agent useful in treating arthritis such as, for example, cyclosporine, azathioprine, lefunomide, methotrexate, glucocorticoid, penicillamine, or hydroxychloroquine.

The compounds disclosed herein possess QR2 IC₅₀ values similar to those of known antimalarial drugs, such as chloroquine, mefloquine and primaquine. In particular, chloroquine has an QR2 IC₅₀ value of roughly 1-5 μM. The quinoline and quinazoline derivatives of this invention displayed QR2 IC₅₀ values of between 0.5-238 μM. See Example 1, herein below, which lists the QR2 IC₅₀ results for several derivatives of the invention.

By “IC₅₀” is intended the concentration of a compound required to inhibit the binding of a ligand by 50%. IC₅₀ values can be determined by binding or activity assays known in the art. See, for example, U.S. Patent Publication 20030143645, and WO 00/63694, both of which are incorporated herein in their entirety by reference.

Methods are provided to inhibit QR2 activity using the compounds of the invention. A decrease in the activity in the presence of the compound is indicative of inhibition. According to the present invention, QR2 activity may be inhibited by at least 10%, at least 20%, at least 30%, at least 40%, at least 50%, at least 60%, at least 70%, at least 80%, at least 90%, or at least 95%. By “QR2 activity” is intended the enzymatic or biological activity of the QR2 enzyme. Inhibition of QR2 activity may be determined by measuring the reductase activity of QR2 in the presence of an inhibitor, or by measuring molecular or biological processes affected by the level of QR2 activity. Inhibition by the compounds of the invention can be assessed by standard techniques known in the art, including binding assays, enzymatic activity assays, cellular proliferation assays, etc. See, for example Jaiswal et al. (1990) Biochemistry 29:1899-906, Jaiswal (1994) J. Biol. Chem. 269:14502-508; and Zhao et al. (1997) Proc. Natl. Acad. Sci. USA 94:1669-1674).

The present invention provides methods of treating malaria or treating an immune disorder. By a “treatment” of a condition is intended any mitigation or reduction of at least one symptom associated with the condition to be treated. The methods comprise the step of administering a pharmaceutical composition comprising one of more compounds of formulas 1-13 to the subject. An effective amount of the compound is administered to the subject. By “effective amount” or a “therapeutically effective amount” of a compound is intended an amount sufficient to treat a condition, i.e. an amount sufficient to mitigate or reduce at least one symptom of the condition to be treated.

Preferred unit dosage formulations are those containing a therapeutically effective amount, or an appropriate fraction thereof, of the active agent of the present invention. The term therapeutically effective amount, as used herein, is meant to refer to an amount effective to treat the disease of interest, such as malaria or an immune disorder.

The compounds of the present invention are generally administered at a dosage of about from about 0.1 to 50 mg/kg body weight, such as about 0.5 to 25 mg/kg body weight, for example about 1 to 20 mg/kg body weight.

The following Examples illustrate the present invention but should not be construed as a limitation to the scope thereof.

### EXAMPLES

### Example 1

### Inhibition of QR2 Activity in vitro

Inhibition of QR2 activity by the compounds of the invention was assayed in triplicate with recombinant QR2 (at 96 ng/ml) by measuring the absorbance at 365 nm in a buffer containing 50 Mm Tris-HCl, Ph 8.5, 50 μM NmeH, 20 μM menadione, and 0.1% Triton X-100 as described in Graves et al. (2002) Mol. Pharmacol. 62: 1364-72.

The following table shows the IC₅₀ values for the inhibition of QR2 by the compounds of the invention. Inhibition of QR2 with chloroquine, mefloquine and primaquine is also shown.

| | Assay Range | | | Std. |
|---|---|---|---|---|
| Compound | (μM) | IC₅₀ (μM) | Average | Dev. |
| chloroquine | 0.05-100 | 10.3 | 1.13 | 0.18 |
| (set 1) | 0.23-500 | 3.4 | | |
| | 0.05-100 | 1.3 | | |
| | 0.05-100 | 1.14 | | |
| | 0.05-100 | 0.951 | | |
| chloroquine | 0.54-872 | 4.15 | 4.13 | 0.13 |
| (set 2) | 0.05-88 | 4.25 | | |
| | 0.05-88 | 3.99 | | |
| mefloquine | 0.52-832 | 28 | 33 | 4 |
| | 0.52-832 | 34.8 | | |
| | 0.21-332 | 36.5 | | |
| primaquine | 0.61-988 | 14.2 | 13 | 2 |
| | 0.61-988 | 11.0 | | |
| | 0.25-395 | 13.2 | | |
| 2-39 | | 0.13 | 0.13 | 0.03 |
| | | 0.16 | | |
| | | 0.09 | | |
| 2-4 | 0.48-767 | 3.3 | 3.8 | 0.8 |
| | 0.05-76 | 3.3 | | |
| | 0.05-76 | 4.7 | | |
| 2-5 | 0.58-927 | 2.68 | 2.7 | 0.2 |
| | 0.02-33 | 2.42 | | |
| | 0.02-33 | 2.86 | | |
| 2-17 | 0.46-741 | 0.41 | 0.60 | 0.17 |
| (set 1) | 0.05-74 | 0.67 | | |
| | 0.05-74 | 0.72 | | |
| 2-17 | | 0.60 | 0.55 | 0.03 |
| (set 2) | | 0.50 | | |
| | | 0.60 | | |
| 2-29 | | 4.1 | 3.2 | 0.78 |
| | | 2.2 | | |
| | | 3.4 | | |
| 2-33 | | 84.0 | 74.0 | 7.5 |
| | | 72.2 | | |
| | | 65.9 | | |
| 2-34 | | 81.5 | 79.6 | 1.4 |
| | | 79.0 | | |
| | | 78.3 | | |
| 4-9 | | 11.6 | 18.5 | 6.6 |
| | | 27.5 | | |
| | | 16.4 | | |
| 3-3 | 0.57-923 | 109 | 98 | 16 |
| | 0.57-923 | 87 | | |
| 3-4 | 0.47-748 | 229 | 238 | 78 |
| | 0.47-748 | 164 | | |
| | 0.19-300 | 320 | | |
| 4-1 | | 0.34 | 0.47 | .26 |
| | | 0.24 | | |
| | | 0.84 | | |
| 4-2 | 0.58-927 | 4.5 | 4.8 | 0.4 |
| | 0.06-92 | 4.7 | | |
| | 0.06-92 | 5.3 | | |
| 4-3 | 0.47-754 | 9.1 | 10.2 | 3.1 |
| | 0.05-75 | 7.8 | | |
| | 0.05-75 | 13.7 | | |
| 4-5 | | 18.5 | 23.0 | 4.3 |
| | | 21.7 | | |
| | | 28.8 | | |
| 4-8 | | 2.2 | 1.6 | 0.52 |
| | | 0.89 | | |
| | | 1.65 | | |
| 4-11 | 0.56-898 | 0.99 | 0.85 | 0.15 |
| | 0.06-90 | 0.69 | | |
| | 0.06-90 | 0.86 | | |
| 5 | 0.32-518 | 132 | 189 | 62 |
| | 0.32-518 | 180 | | |
| | 0.13-207 | 255 | | |
| 7 | 1.3-2085 | 49.5 | 43.3 | 5.4 |
| | 0.43-695 | 40 | | |
| | 0.43-695 | 40.3 | | |
| 9 | 0.01-11.5 | 4.2 | 4.9 | 0.8 |
| | 0.07-114 | 4.7 | | |
| | 0.07-114 | 5.8 | | |

### Inhibition of QR2 Activity in vivo

The AS line of Plasmodium chabaudi, a synchronous parasite which is sensitive to all currently used antimalarial drugs, has been used as an indicator of baseline response to CQ derivatives of this invention. To determine the effect of compound 2-39 against resistant strains of rodent malaria, two lines of P. yoelii were employed. These were the inherently chloroquine-resistant P. yoelii ssp. NC, which is a valuable model for naturally occurring chloroquine-resistant P. falciparum (Peters et al., Annals of Tropical Medicine and Parisitology, 69, 155-171 (1975)) and the mefloquine-resistant P. yoelli ssp. MEFA which was developed from NS by the 2% relapse technique (Peters, W., Chemotherapy and Drug Resistance in Malaria, 2^{nd} Ed., London, Academic Press, ISBN 0-12-552721-7).

Blood schizontocidal activity was assessed by the “four day test” essentially as described by Peters, W. and Robinson, B. L. (1999), “Malaria” in Handbook of Animal Models of Infection, Academic Press. The compound was dissolved in dimethyl sulfoxide with the aid of ultrasonification and aqueous dilutions were prepared for use.

Random-bred Swiss albino mice free of Mycoplasma (Eperythrozoon) coccoides and weighing between 18 and 22 g. were infected via the tail vein on day 0 (D0) and then treated once daily either subcutaneously or orally by gavage for four consecutive days (D0 through D+3). Thin blood films were made from tail blood from groups of untreated controls and from treated animals on D+4. Levels of parasitaemia, as seen in Giemsa stained smears, were assessed and 50% and 90% activities (ED50, ED90) were determined graphically from plots of log-dose/probit activity prepared using Microcal Origins®.

Compound 2-39 was active both parenterally and orally against the drug-sensitive P. chabaudi AS line (see table below), validating QR2 as a target. The compound also demonstrated inhibitory activity against the two resistant strains of P. yoelii. No overt signs of toxicity were observed in mice that received compound 2-39 subcutaneously, but some toxicity was observed at higher doses when the compound was administered orally.

TABLE 2
Summary of Results Obtained from 4 day
test for Blood Schizontocidal Activity

| | | 2-39 | | Chloroquine | | Mefloquine | |
|---|---|---|---|---|---|---|---|
| Parasite | Route | ED50 | ED90 | ED50 | ED90 | ED50 | ED90 |
| P. chabaudi AS | sc* | 150 | 500 | 1.8 | 3.2 | 1.5 | 3.0 |
| | po** | 150 | 310 | — | — | — | — |
| P. yoelii ssp. NS | sc | 400 | 4000 | 5.0 | 19.0 | — | — |
| | po | 220 | 550 | — | — | — | — |
| P. yoelii ssp. MEFA | sc | 320 | 5500 | — | — | 20.0 | 100 |
| | po | 190 | 300 | — | — | — | — |

*sc - indicates subcutaneous injection
**po - indicates oral administration

All publications, patents and patent applications mentioned in the specification are indicative of the level of those skilled in the art to which this invention pertains. All publications, patents and patent applications are herein incorporated by reference to the same extent as if each individual publication, patent or patent application was specifically and individually indicated to be incorporated by reference.

Although the foregoing invention has been described in some detail by way of illustration and example for purposes of clarity of understanding, it will be obvious that certain changes and modifications may be practiced within the scope of the embodiments.

That which is claimed:

1. A compound of the formula 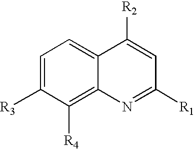
wherein R₁ is H or trifluoromethyl;
R₂ is NHR₅, NR₅R₆, OR₅, SR₅;
R₃ is H, Cl, or trifluoromethyl;
R₄ is H or trifluoromethyl;
R₅ is alkyl, allyl, propargyl, aryl, substituted aryl, heteroaryl, heteroarylalkylamino, heteroarylalkyl, substituted heteroaryl, cycloalkyl optionally substituted with aryl, substituted or nonsubstituted heterocycloalkyl, C₁₋₂ alkyl optionally substituted with aryl, C₁₋₂ alkyl optionally substituted with mono- or di-substituted aryl, C₁₋₂ alkyl optionally substituted with substituted or nonsubstituted heteroaryl, C₁₋₂ alkyl optionally substituted with substituted or nonsubstituted cycloalkyl, C₁₋₂ alkyl optionally substituted with substituted or nonsubstituted heterocycloalkyl, C₁₋₃ alkyl optionally substituted with aminoalkyl, amido, aminoalkoxy, aminoheteroaryl, hydroxy, alkoxy, C₁₋₃ alkyamino optionally substituted with C₁₋₃ hydroxy; and
R₆ is methyl;
or a pharmaceutically acceptable ester, amide, salt, or solvate thereof.

2. A compound according to claim 1, wherein the compound is of the formula 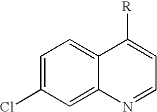
wherein R is selected from the group consisting of (3-methoxyphenyl) methylamino, (4,4-diethoxy)butylamino, isopentylamino, 2-(pyridin-2-yl)ethylamino, 2-(4-hydroxyphenyl)ethylamino, (2-chloro-4-fluoro)benzylamino, (pyridin-3-yl)methylamino, 3-(dibutylamino)propylamino, 2-(4-(ethoxycarbonyl)phenyl) ethylamino, 4-(hydrazinocarbonyl)phenylamino, naphthalen-1-ylmethylamino, ((1r, 4r)-4(ethoxycarbonyl)cyclohexyl)methylamino, allylamino, prop-2-ynylamino, pyridin-2-ylmethylamino, o-N-toluidinoamino, 4-(morpholino)phenylamino, benzylamino, 2-(acetamido)ethylamino, 1-(pyridin-2-yl)ethylamino, 2-(4-sulfamoylphenyl)ethylamino, (1-hydroxy-3-methyl)but-2-ylamino, (thiophen-2-ylmethyl)amino, 1,2,3,4-tetrahydronaphthalen-1-ylamino, 3a, 7a-dihydro-1H-benzo[d]imidazol-2-ylamino, (3-methoxyphenylethyl)amino, (6-methylpyridin-2-yl)methoxy, (pyridin-2-ylmethyl)thio, pyridin-2-ylmethoxy, N-methyl-N-(pyridin-2-ylmethyl)amino, and 1-(pyridin-2 yl)ethoxy;
or a pharmaceutically acceptable ester, amide, salt, or solvate thereof.

3. A compound according to claim 1, wherein the compound is of the formula 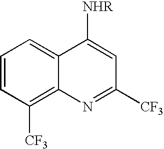
wherein R is selected from the group consisting of 2-(pyrrolidin-1-yl)ethyl, 2-(4-hydroxyphenyl)ethyl, 2-(2-hydroxypropylamino)ethyl, 3-(bis(2-hydroxyethyl)amino) propyl, 1-benzylpiperidin-4-yl, 2-(thiophen-2-yl)ethyl, 1-(4-fluorophenyl)ethyl, and 2-(pyridin-2-yl)ethyl;
or a pharmaceutically acceptable ester, amide, salt, or solvate thereof.

4. A compound according to claim 1, wherein the compound is of the formula 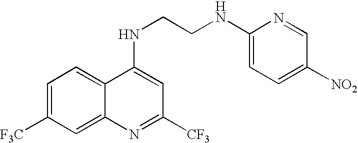
or a pharmaceutically acceptable ester, amide, salt, or solvate thereof.

5. A pharmaceutical composition comprising one or more compounds according to claim 1.

6. A compound of the formula 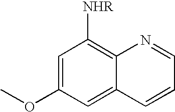
wherein R is selected from the group consisting of pyridin-2-ylmethyl, 1-benzylpiperidin-4-yl, 4-cyano-2,2-diethylbutyl, 2-chlorocyclopentyl, 4-(diethylamino)butan-2-yl, 1-(furan-2-yl)ethyl, 1-cyclopropylethyl, 1-ethylpiperidin-4-yl, 5-amino-2,2-diethylpentyl, and 2-(diethylphosphoryl)-1-methylethyl;
or a pharmaceutically acceptable ester, amide, salt, or solvate thereof.

7. A pharmaceutical composition comprising one or more compounds according to claim 6.

8. A compound of the formula: 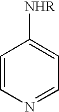
wherein R is selected from the group consisting of furan-2-ylmethyl, pyridin-3-ylmethyl; and pyridin-4-ylmethyl;
or a pharmaceutically acceptable ester, amide, salt, or solvate thereof.

9. A pharmaceutical composition comprising one or more compounds according to claim 8.

10. A method for inhibiting the activity of QR2, comprising contacting QR2 with one or more compounds of the formula: 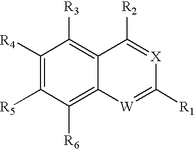
wherein W is N or N⁺O⁻;
X is CR₁₄ or N;
R₁ is H or trifluoromethyl;
R₂ is NR₇R₈, OR₁₁, SR₁₂, or alkyl;
R₃is H or OR₁₃;
R₄ is H or methoxy;
R₅ is H, Cl, or trifluoromethyl;
R₆ is H, NR₉R₁₀ or trifluoromethyl;
R₇ is H, C₁₋₅ alkyl, heteroarylalkyl, cycloalkyl, heterocycloalkyl, aryl, heteroaryl, ureido, thioureido, alkenyl, alkynyl, amido, amino, alkoxy, alkylamino, alkylphosphonate, alkylnitrile, alkylhalo, or alkylhalo optionally substituted with C₁₋₅ alkyl, cycloalkyl, cycloalkenyl, heterocycloalkyl, CO-1 aryl, heteroaryl, alkenyl, alkynyl, amido, alkoxy, alkylamino, alkylhydroxy, halo, hydroxyl, carboxylate, alkylcarboxylate, acylazido, sulfonamide or alkyl halo;
R₈ is H, C₁₋₅ alkyl, cycloalkyl, heterocycloalkyl, aryl, heteroaryl, ureido, thioureido, alkenyl, alkynyl, amido, amino, alkoxy, alkylamino, alkylphosphonate, alkylnitrile, alkylhalo or alkylhalo optionally substituted with C₁₋₅ alkyl, cycloalkyl, cycloalkenyl, heterocycloalkyl, C0-1 aryl, heteroaryl, alkenyl, alkynyl, amido, alkoxy, alkylamino, alkylhydroxy, halo, hydroxyl, carboxylate, alkylcarboxylate, acylazido, sulfonamide or alkylhalo;
R₉ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, aryl, heteroaryl or alkylamino;
R₁₀ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, heteroaryl, or alkylamino;
R₁₁ is alkyl, aryl or heteroaryl optionally substituted with alkyl, haloalkyl, aryl or heteroaryl;
R₁₂ is alkyl, aryl or heteroaryl optionally substituted with alkyl, haloalkyl, aryl or heteroaryl;
R₁₃ is alkyl or aryl optionally substituted with alkyl or haloalkyl; and
R₁₄ is H or aryl;
or a pharmaceutically acceptable ester, amide, salt, or solvate thereof.

11. The method of claim 10, wherein the contacting is performed in vitro.

12. The method of claim 10, wherein the contacting is performed in vivo.

13. The method of claim 10, comprising contacting QR2 with a compound of the formula 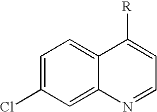
wherein R is selected from the group consisting of (pyridin-2-yl)methylamino, (3-methoxyphenyl) methylamino, (4,4-diethoxy)butylamino , isopentylamino, 2-(pyridin-2-yl)ethylamino, 2-(4-hydroxyphenyl)ethylamino, (2-chloro-4-fluoro)benzylamino, (pyridin-3-yl)methylamino, 3-(dibutylamino)propylamino, 2-(4-(ethoxycarbonyl)phenyl) ethylamino, 4-(hydrazinocarbonyl)phenylamino, naphthalen-1-ylmethylamino, 2,2-diphenylpropylamino, ((1r, 4r)-4-(ethoxycarbonyl)cyclohexyl) methylamino, 3-chloropropylamino, allylamino, prop-2-ynylamino, pyridin-2-ylmethylamino, (2,2-dimethyl-3-dimethylamino)propylamino, o-N-toluidinoamino, 4-(morpholino)phenylamino, benzylamino, 2-(acetamido)ethylamino, 1-(pyridin-2-yl)ethylamino, 2-(4-sulfamoylphenyl)ethylamino, (1-hydroxy-3-methyl)but-2-ylamino, (thiophen-2-ylmethyl)amino, morpholinoamino, 1,2,3,4-tetrahydronaphthalen-1-ylamino, 3a, 7a-dihydro-1H-benzo[d]imidazol-2-ylamino, (3-methoxyphenylethyl)amino, 2-(cyclohexenyl)ethylamino, (6-methylpyridin-2-yl)methoxy, (pyridin-2-ylmethyl)thio, pyridin-2-ylmethoxy, N-methyl-N-(pyridin-2-ylmethyl)amino, 1-(pyridin-2-yl)ethoxy, 3-(2-carboxy)naphthylamino, 4-(2,4-dichlorophenyl)thiosemicarbazido, and methylamino;
or a pharmaceutically acceptable ester, amide, salt, or solvate thereof.

14. The method of claim 10, comprising contacting QR2 with a compound of the formula 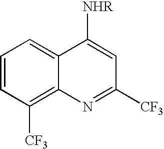
wherein R is selected from the group consisting of 2-(pyrrolidin-1-yl)ethyl, 2-(4-hydroxyphenyl)ethyl, 2-(2-hydroxypropylamino)ethyl, 3-(bis(2-hydroxyethyl)amino) propyl, 1-benzylpiperidin-4-yl, 2-(thiophen-2-yl)ethyl, 1-(4-fluorophenyl)ethyl, and 2-(pyridin-2-yl)ethyl;
or a pharmaceutically acceptable ester, amide, salt, or solvate thereof.

15. The method of claim 10, comprising contacting QR2 with a compound of the formula 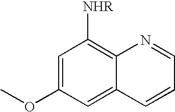
wherein R is selected from the group consisting of pyridin-2-ylmethyl, 1-benzylpiperidin-4-yl, 4-cyano-2,2-diethylbutyl, 2-chlorocyclopentyl, 4-(diethylamino)butan-2-yl, 1-(furan-2-yl)ethyl, 1-cyclopropylethyl, 1-ethylpiperidin-4-yl, 5-amino-2,2-diethylpentyl, and 2-(diethylphosphoryl)-1-methylethyl;
or a pharmaceutically acceptable ester, amide, salt, or solvate thereof.

16. The method of claim 10, comprising contacting QR2 with a compound of the formula 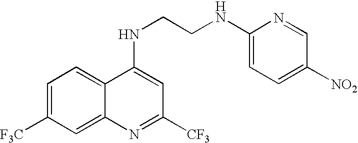
or a pharmaceutically acceptable ester, amide, salt, or solvate thereof.

17. The method of claim 10, comprising contacting QR2 with a compound of the formula 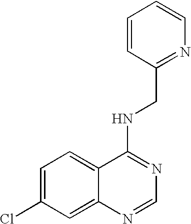
or a pharmaceutically acceptable ester, amide, salt, or solvate thereof.

18. The method of claim 10, comprising contacting QR2 with a compound of the formula 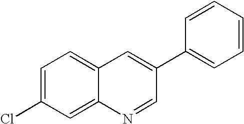
or a pharmaceutically acceptable ester, amide, salt, or solvate thereof.

19. The method of claim 10, comprising contacting QR2 with a compound of the formula 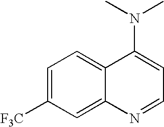
or a pharmaceutically acceptable ester, amide, salt, or solvate thereof.

20. The method of claim 10, comprising contacting QR2 with a compound of the formula 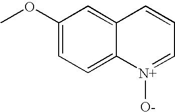
or a pharmaceutically acceptable ester, amide, salt, or solvate thereof.

21. The method of claim 10, comprising contacting QR2 with a compound of the formula 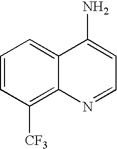
or a pharmaceutically acceptable ester, amide, salt, or solvate thereof.

22. A method for inhibiting the activity of QR2, comprising contacting QR2 with a compound of the formula 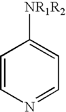
wherein R₁ is H, alkyl, aryl or heteroaryl optionally substituted with aryl or heteroaryl and R₂ is H, alkyl, aryl or heteroaryl optionally substituted with aryl or heteroaryl;
or a pharmaceutically acceptable ester, amide, salt, or solvate thereof.

23. The method of claim 22, wherein the contacting is performed in vitro.

24. The method of claim 22, wherein the contacting is performed in vivo.

25. A method for inhibiting the activity of QR2, comprising contacting QR2 with a compound of the formula 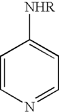
wherein R is a Cl alkyl optionally substituted with aryl or heteroaryl;
or a pharmaceutically acceptable ester, amide, salt, or solvate thereof.

26. The method of claim 25, wherein R is selected from the group consisting of thiophen-2-ylmethyl, furan-2-ylmethyl, pyridin-3-ylmethyl, and pyridin-4-ylmethyl.

27. The method of claim 25, wherein the contacting is performed in vitro.

28. The method of claim 25, wherein the contacting is performed in vivo.
